(19) United States
(12) Patent Application Publication
(10) Pub. No.: US 2014/0227227 A1
(43) Pub. Date: Aug. 14, 2014

(54) USE OF ROSEBURIA IN THE PREVENTION AND TREATMENT FOR OBESITY RELATED DISEASES

(71) Applicants: Junjie Qin, Shenzhen (CN); Shenghui Li, Shenzhen (CN); Jianfeng Zhu, Shenzhen (CN); Xiaoping Li, Shenzhen (CN); Suisha Liang, Shenzhen (CN); Jun Wang, Shenzhen (CN); Jian Wang, Shenzhen (CN); Huanming Yang, Shenzhen (CN); Qiang Feng, Shenzhen (CN)

(72) Inventors: Junjie Qin, Shenzhen (CN); Shenghui Li, Shenzhen (CN); Jianfeng Zhu, Shenzhen (CN); Xiaoping Li, Shenzhen (CN); Suisha Liang, Shenzhen (CN); Jun Wang, Shenzhen (CN); Jian Wang, Shenzhen (CN); Huanming Yang, Shenzhen (CN); Qiang Feng, Shenzhen (CN)

(73) Assignee: BGI Shenzhen Co., Limited, Shenzhen Guangdong (CN)

(21) Appl. No.: 13/497,043

(22) PCT Filed: Dec. 22, 2011
(86) PCT No.: PCT/CN2011/084450

§ 371 Date: Jul. 17, 2013

(30) Foreign Application Priority Data
Nov. 2, 2011 (CN) 201110342339.6

(51) Int. Cl. A61K 35/74; A23L 1/30

(52) U.S. Cl. CPC A61K 35/74; A23L 1/3014
USPC 424/93.4

(57) ABSTRACT

The present invention provides use of Roseburia in the preparation of composition for prevention and/or treatment of obesity related diseases. Also provided are a composition used in the treatment and prevention for obesity related diseases, comprising pharmaceutical, drink, food, and/or animal feed composition, etc; and a method of reducing body weight and/or blood glucose.

### TECHNICAL FIELD

The invention relates to microbiology, specifically, this invention relates to Roseburia bacterial strains in the treatment and prevention of obesity-related diseases in the application, and also involves the composition comprising Roseburia bacteria and its application.

### BACKGROUND

Obesity is a chronic disease, and many factors can lead to obesity. The incidence of origin is not clear so far. Obesity is also a factor for a range of diseases, such as hypertension, diabetes, coronary heart disease, gallbladder disease, osteoarthritis, sleep apnea, respiratory disorders, uterus tumor, prostate cancer, breast cancer and colon cancer. According to NIH's report, about 97 million Americans are overweight and suffer from obesity, including about 15.1 million people suffering from obesity associated with type II diabetes. Each year nearly 200 thousand people die from obesity-related diseases.

Obesity is usually caused by changes in physiological or biochemical function which lead to excess body fat. Fat generally includes neutral lipid, phospholipid and cholesterol. Fat increase is due to that energy intake is greater than energy consumption. According to pathogenesis, there are two types of obesity: (a) simple obesity and (b) second obesity. Simple obesity can be divided into idiopamic obesity and acquired obesity. The number of simple obesity patients may be more than 95% of the total number of obesity. Idiopamic obesity is caused by a large number of fat cells, and is common in childhood obesity. Acquired obesity is due to larger fat cells, and is common in adult obesity. Second obesity is also known as symptomatic obesity, which is usually caused by endocrine or metabolic diseases.

There are five treatment strategies for obesity: diet, exercise, behavior therapy, drug treatment and therapeutic operation. Which strategy to be chosen depends on patients' health risk factors and the speed and effectiveness of weight loss. Combination of these strategies can also be used for the treatment of obese patients. The speed and effectiveness of their weight loss are influenced by many factors such as age, height, family history and risk factors. Diet-exercise therapy is to eat low-calorie, low fat foods combined with aerobic exercise, and requires long-term adherence to be effective. Thus, this strategy is generally considered to be not successful to the general public. Surgical removal of body fat can achieve immediate results, but there are many restrictions, such as surgical risk, effects which are difficult to sustain and too expensive cost.

Drug therapy is the main clinical treatment of obesity and its major obesity-related diseases (such as diabetes) method. Mechanism of drug therapy includes restraining appetite, increasing energy consumption, stimulating fat movement, reducing triglyceride synthesis and inhibiting fat absorption. The main drugs at present are phenylpropanolamine (PPA), orlistat (Xenical III) and sibutramine (Reductil™). Some people, who have diabetes still cannot properly control high blood glucose through diet and/or exercise therapy or the use of such therapeutic compounds, should use supplemental insulin. To these patients, the use of supplemental insulin is very expensive and a painful way, which causes multiple complications. For example, the absence of meals or normal exercise and insulin dose calculation errors can lead to insulin response (low blood sugar). In addition, the use of drugs may also occur on the local or systemic drug allergies or immune resistance.

At present there is still no method or drug, which is effective and has fewer side effects, for treatment and prevention of obesity and related diseases. So there is an urgent need to develop a new, non-toxic drug for the treatment and prevention of obesity and its related diseases in this field.

### SUMMARY

One aim of the present application is to provide the use of Roseburia in the prevention and treatment of obesity related diseases.

Another aim of the present application is to provide pharmaceutical, drink, food composition and/or animal feed composition in the prevention and treatment of obesity related diseases.

The third aim of the present application is to provide a method of reducing body weight and/or blood glucose and application thereof.

According to the first aspect of this application, provided is use of Roseburia in the preparation of the composition for treatment and/or prevention of obesity.

In one preferred embodiment, the composition is selected from the group of pharmaceutical composition, food composition, drink composition, and feed composition.

In another preferred embodiment, the Roseburia is Roseburia inulinivorans.

In another preferred embodiment, the Roseburia is Roseburia inulinivorans DSM 16841.

In another preferred embodiment, the composition is used in improving the tolerance of blood glucose.

According to the second aspect of the present application, provided is a food composition, the food composition comprises an effective amount of Roseburia and/or the metabolite thereof and a carrier acceptable for food compositions.

In another preferred embodiment, the forms of the food composition is selected from granule, capsule, tablet, powder agent, oral liquid, suspension, and/or emulsion.

In another preferred embodiment, the food composition is used in reducing body weight and/or blood glucose, and improving the tolerance of blood glucose.

In another preferred embodiment, the effective amount is Roseburia of 1×10-1×10²⁰ cfu/mL (or cfu/g), preferably 1×10⁴-1×10¹⁵ cfu/mL (or cfu/g).

According to the third aspect of the present application, provided is a pharmaceutical composition, the pharmaceutical composition comprises an effective amount of Roseburia, its metabolite thereof, and/or the pharmaceutically acceptable carrier.

In another preferred embodiment, the forms of the pharmaceutical composition is selected from granule, capsule, tablet, powder agent, oral liquid, suspension, and/or emulsion.

In another preferred embodiment, the pharmaceutical composition is used in reducing body weight and/or blood glucose, and improving the tolerance ability of blood glucose.

In another preferred embodiment, the effective amount is Roseburia of 1×10-1×10²⁰ cfu/mL (or cfu/g), preferably 1×10⁴-1×10¹⁵ cfu/mL (or cfu/g).

In one preferred embodiment, the food composition according to the second aspect or the pharmaceutical composition according to the third aspect comprises: Roseburia of 1×10-1×10²⁰ cfu/mL, a pharmaceutically acceptable carrier or a carrier acceptable for food compositions, and/or excipient.

In one preferred embodiment, the food composition according to the second aspect or the composition according to the third aspect comprises: Roseburia of 1×10⁴-1×10¹⁵ cfu/mL, a pharmaceutically acceptable carrier or a carrier acceptable for food compositions, and/or excipient.

In one preferred embodiment, the food composition according to the second aspect or the pharmaceutical composition according to the third aspect comprises: Roseburia of 1×10⁶-1×10¹¹ cfu/mL, a pharmaceutically acceptable carrier or a carrier acceptable for food compositions, and/or excipient.

According the fourth aspect of the present application, provided is the use of the food composition according to the second aspect or the pharmaceutical composition according to the third aspect, the composition is used for reducing the blood glucose and/or body weight of subjects.

In one preferred embodiment, the composition is used for improving the tolerance of blood glucose.

According the fifth aspect of the present application, provided is a method of reducing body weight and/or blood glucose, comprising: feeding or applying the Roseburia according to the first aspect of the present application, the food composition according to the second aspect, and/or the pharmaceutical composition according to the third aspect to subjects in need.

According the sixth aspect of the present application, provided is use of Roseburia in preparation of compositions for improving the tolerance of blood glucose.

According the seventh aspect of the present application, provided is a method of improving the tolerance of blood glucose, comprising: administering the Roseburia according to the first aspect of the present application, the food composition according to the second aspect, and/or the pharmaceutical composition according to the third aspect to subjects in need. According the eighth aspect of the present application, provided is a method of prevention and/or treatment of obesity related diseases, comprising: administering the Roseburia according to the first aspect of the present application, the food composition according to the second aspect, and/or the pharmaceutical composition according to the third aspect to subjects in need. In another preferred embodiment, the obesity related diseases are selected from the group consisting of diabetes, hypertension, hyperglycemia, hyperlipidemia, coronary heart disease, atherosclerosis, stroke, or the combination thereof.

According to all aspects of the present application, the subjects is preferable a mammal, more preferably mice or humans. It should be understood within the scope of the present application, the above-mentioned technical features and technical features mentioned in the embodiments below can be combined freely and mutually to form new or preferable embodiments, even if such combinations are not explicitly described herein.

### BRIEF DESCRIPTION OF THE DRAWING

FIG. 1 illustrates the grouping information and steps of the treatment in the embodiments.

FIG. 2 illustrates the increase tendency of the body weight of three groups of mice within 10 weeks after feeding.

FIG. 3 illustrates the significance (P value) of the differences in the increase of the body weight among three groups of mice.

FIG. 4 illustrates the changing curve of the blood glucose of the mice in 10th week after feeding.

FIG. 5 illustrates the tolerance of blood glucose of treated mice in the 10th week after feeding.

### DESCRIPTION OF THE EMBODIMENTS

With extensive experiments and in-depth research, it is surprisingly discovered the use of Roseburia in prevention and/or treatment of obesity-related diseases. After applying the active composition comprising Roseburia to experimental subjects, it is surprisingly discovered that the composition is capable of inhibiting body weight growth, reducing blood glucose, improving the tolerance ability of blood glucose, ameliorating diabetes and obesity effectively. The present invention is based on this discovery.

As used herein, the term “comprise” or “comprising” indicates all components can be used in the mixture or composition in the present application. So the meaning of “mainly composed of”, “consist mainly of”, and “consisted of” belongs to the scope of the term “comprise” or “comprising”.

As used herein, the term “Roseburia bacterial strain” and “Roseburia” are interchangeably used. In a preferred embodiment, the bacterial strain is Roseburia inulinivorans DSM 16841, purchased from DSMZ (Deutsche Sammlung von Mikroorganismen and Zellkulturen GmbH). The physiological properties of Roseburia are as follows: slightly curved rod-shaped bacteria, about 0.5 μm×2.3˜5.0 μm in size, does not form spores, motile, has clusters of flagella at the proximal end of the concave of the bacterial cell, capable of fermenting carbohydrates include wood sugar, galactose, raffinose, glucose, maltose, cellobiose, sucrose, starch and glycogen, mainly produces or only produces butyric acid. The cultivation conditions are: in M2GSC media, at 37° C., anaerobic, for 2-5 days.

The present application discloses the use of Roseburia in prevention and/or treatment of obesity and related diseases. The bacterial strain DSM 16841 has the property of inhibiting the growth of the body weights of subjects after their intake of high-fat food. According to one embodiment of the present application, the CF57BL/6J mice that are fed with high-fat food that will induce obesity and treated with the bacterial strain DSM 16841 will retain body weight compared with the untreated control group. Such treatment is also beneficial to reducing blood glucose level. Therefore, the bacterial strain can be applied to prevent or treat obesity and related diseases, e.g., diabetes. etc.

The present application further provides a composition, preferably a pharmaceutical composition. The composition comprises an effective amount of the Roseburia bacterial strain. In one preferable embodiment, the composition further comprises the bacterial strains selected from the group consisting of: Lactobacillus gasseri, Lactobacillus rhamnosus GM-020, Lactobacillus rhamnosus PL60 and/or the combination thereof.

The pharmaceutical compositions can be administrated in forms of tablet, capsule and/or injection. These pharmaceutic compositions comprise excipients, pharmaceutically acceptable media and/or carriers. Appropriate excipients, media and/or carriers may be selected depending on the routes of administration. The pharmaceutical compositions in the present application can further comprise auxillary active constituents.

The carrier, excipient, and/or thinner comprises but is not limited to lactose, glucose, sucrose, sorbitol, mannose, starch, gum arabic, calcium phosphate, alginate, gelatin, calcium silicate, fine crystalline cellulose, polyvinyl pyrrolidone (PVP), cellulose, water, syrup, methyl cellulose, methyl p-hydroxybenzoate, propyl para-hydroxybenzoate esters, talc, magnesium stearate or mineral oil, etc.

The pharmaceutical composition can further comprise lubricant, wetting agent, emulsifier, suspension stabilizer, preservative, sweetener, spices, etc. The pharmaceutical composition in the present application can be manufactured so that the active constituent of the pharmaceutical composition, i.e., the bacterial strains contained therein can be protected from gastric acid and go through the stomach intact by the various methods known in the art.

Furthermore, microorganisms of the present invention may be administered in a form of capsule prepared by conventional process. For example, standard excipient and lyophilized microorganisms of the present invention are mixed together and prepared to pellets and then, the pellets are filled into hard gelatin capsules. In addition, the microorganisms of the present invention and pharmaceutically allowable excipient, for example, aqueous gum, cellulose, and/or oil, are mixed to produce a suspension or emulsion and then, this suspension or emulsion may be filled into soft gelatin capsule.

The pharmaceutical composition of the present application may be prepared as an enterically coated tablets or capsules for oral administration. The term “the enteric coating” of this application includes all conventional pharmaceutically acceptable coating that has resistance to gastric juice, but can disintegrate sufficiently in the small intestines for a rapid release of the microorganisms of the present invention. The enteric coating of the present invention can be maintained for more than 2 hours in synthetic gastric juice, such as HCl solution of pH 1 at the temperature of 36-38° C., and preferably decomposes within 1.0 hours in synthetic intestinal juice, such as buffer solution of pH 7.0.

The enteric coating of the present invention applies to each tablet with the amount of about 16 to 30 mg, desirably 16 to 25 mg, more desirably 16 to 20 mg. The thickness of enteric coating of the present invention is 5 to 100 μm, desirably 20 to 80 μm. The components of the enteric coating are selected appropriately from commonly known polymeric materials.

The preferred enteric coating of the present invention are prepared from polymers of cellulose acetate phthals or trimelitated and methacrylic copolymer (for example, copolymer of more than 40% of methacrylic acid and methacrylic acid which contains hydroxyprophyl methylcellulose phthalate or derivatives from ester thereof).

Cellulose acetate phthalate employed in the enteric coating of the present invention, has about 45 to 90 cP of viscosity, 17% to 26% of acetyl contents and 30% to 40% of phthalate contents. The cellulose acetate trimelitate used in the enteric coating, has about 15 to 21 cs of viscosity, 17% to 26% of acetyl contents. The cellulose acetate trimelitate manufactured by the Eastman Kodak Company may be used as a material for the enteric coating of the present invention.

Hydroxyprophyl methylcellulose phthalate used in the enteric coating of the present invention has molecular weight of generally 20,000 to 130,000 dalton, desirably 80,000 to 100,000 dalton and has 5% to 10% of hydroxyprophyl cotents, 18% to 24% of metoxy contents, and 21 to 35% of phthalyl contents.

Hydroxyprophyl methylcellulose phthalate used in the enteric coating of the present invention is HP50 manufactured by the Shin-Etsu Chemical Co. Ltd., Japan. The HP50 has 6% to 10% of hydroxyprophyl contents, 20% to 24% of metoxy contents, 21% to 27% of prophyl contents, and molecular weight is 84,000 dalton. Another material for enteric coating manufactured by the Shin-Etsu Chemical Co. Ltd., is HP55. HP55 can also be used as material for the enteric coating of the present invention. The HP55 has 5% to 9% of hydroxyprophyl contents, 18% to 22% of metoxy contents, 27% to 35% of phthalate contents, and molecular weight is 78,000 dalton.

The enteric coating of the present invention is prepared by using conventional methods of spraying the enteric coating solution to the core. Solvents used in the process of the enteric coating are alcohol such as ethanol, ketone such as acetone, halogenated hydrocarbon such as dichloromethane, or the mixture thereof. Softeners such as Di-n-butylphthalate and triacetin are added to the enteric coating solution in the ratio of 1 part coating material to about 0.05 or to about 0.3 part softner. A spraying process is preferably performed continuously, and the amount of materials sprayed may be controlled depending on the condition of the coating process. Spraying pressure may be regulated variously and, generally, desirable result can be obtained under the pressure of average 1 to 1.5 bar.

As used herein, the term “pharmaceutically effective amount” refers to an amount of a compound sufficient to achieve desirable function or activity in the humans and/or animals in need and acceptable to such humans and/or animals. In the present application, the effective amount is 1×10-1×10²⁰ cfu/mL Roseburia, preferably 1×10⁴-1×10¹⁵ cfu/mL, further preferably 1×10⁶-1×10¹¹ cfu/mL.

The effective amount of Roseburia and/or the metabolite thereof in the preparation of the pharmaceutical composition can vary depending on the mode of administration and the severity of obesity of the patient. The dosage form suitable for oral administration comprises Roseburia of 1×10-1×10²⁰ cfu/mL (or cfu/g), preferably 1×10⁴-1×10¹⁵ cfu/mL, further preferably 1×10⁶-1×10¹¹ cfu/mL, which can be mixed with the solid or liquid pharmaceutically acceptable carrier.

The Roseburia and/or the metabolite thereof can be administrated orally and by other specified route suitable for Roseburia and/or its metabolite thereof. The solid carrier comprises starch, lactose, dicalcium phosphate, microcrystalline cellulose, sucrose and white clay, and the liquid carrier comprises media, polyethylene glycol, non-ionic surfactant, and edible oils (such as corn oil, peanut oil, and sesame oil). The adjuvants can be incorporated in the preparation of the pharmaceutical composition advantageously, which comprise: flavoring agents, pigments, preservatives and antioxidants such as vitamin E, vitamin C, BHT and BHA.

In term of preparation and administration, the preferred pharmaceutical composition is solid, further preferably in forms of tablet and capsule with solid or liquid filling. The oral administration is preferred.

The composition of the present invention may be administered once or more per day on the subject. The unit of administration amount means that it is separated physically and thus is suitable for the unit administration for the human subjects and all other mammalian animals. Each unit contains a pharmaceutically acceptable carrier and the microorganisms of the present invention in an effective amount. The administration amount can vary depending on the weight and the severity of obesity of the patient, supplemental active ingredients included and microorganisms used therein. In addition, it is possible to divide up the daily administration amount and to administer continuously, if needed. Therefore, the range of the administration amount does not limit the scope of the present invention. The compositions according to the present invention include not only pharmaceutical compositions, but also food compositions and health supplements. In preferred embodiments, the compositions include beverage, food, drug, animal feed, etc.

In one preferred embodiment, provided is a food composition comprising a effective amount of Roseburia, and/or metabolite thereof, and/or carrier acceptable for food compositions, wherein forms of the food composition is selected from solid, diary, solution, powder, and/or suspension.

In one preferred embodiment, provided is a pharmaceutical composition comprising a pharmaceutically effective amount of Roseburia, and/or metabolite thereof, and/or pharmaceutically acceptable carrier, wherein forms of the pharmaceutical composition is selected from granule, capsule, tablet, powder agent, oral liquid, suspension, and/or emulsion.

The composition comprises: Roseburia of 1×10-1×10²⁰ cfu/mL, its metabolite thereof, a carrier acceptable pharmaceutically or for food compositions, and/or excipient.

In another preferred embodiment, the composition comprises: Roseburia of 1×10⁴-1×10¹⁵ cfu/mL, its metabolite thereof, a carrier acceptable pharmaceutically or for food compositions, and/or excipient.

In another most preferred embodiment, the composition comprises: Roseburia of 1×10⁶-1×10¹¹ cfu/mL, its metabolite thereof, a carrier acceptable pharmaceutically or for food compositions, and/or excipient.

The present application further provides a method of reducing body weight and/or blood glucose, and improving the tolerance of blood glucose. In another preferred embodiment, the method comprises: administering the pharmaceutical composition, food composition, drink composition, and/or the combination thereof to a subject in need thereof. The subject is a human.

In another preferred embodiment, the method comprises: administering the pharmaceutical composition, food composition, drink composition, and/or the combination thereof. The subject is an animal, preferable a mouse or a rabbit.

The advantages of the present application include:
(a) The Roseburia in the present application possesses the excellent ability of prevention for obesity and related diseases;
(b) The Roseburia in the present application possess the ability of treating obesity and related diseases, reducing blood glucose, improving the tolerance ability of blood glucose.

The following exemplary embodiments further describe the present application. Although the description referred to particular embodiments, it will be clear to one skilled in the art that the present application may be practiced with variation of these specific details. Hence this application should not be construed as limited to the embodiments set forth herein. Further, for the embodiments in which details of the experimental methods are not described, such methods are carried out according to conventional conditions such as those described in Sambrook et al., Molecular Cloning: A Laboratory Manual (New York: Cold Spring Harbor Laboratory Pres, 1989), or suggested by the manufacturers.

### Example 1

### Materials

Fifteen C57BL/6J mice were 6 weeks old at the beginning of the experiment and purchased from Laboratory Animal Center of Southern Medical University, China. The mice were fed with a control diet and housed in groups of five per cage in same environment (12-h daylight cycle, lights off at 6 p.m.) with free access to food and water.

Roseburia inulinivorans DSM 16841 was purchased from DSMZ (Deutsche Sammlung von Mikroorganismen and Zellkulturen GmbH).

High-fat diet was purchased from Beijing HFK Bioscience co., LTD, China, containing 58% fat (soybean oil and lard, g/100 g of total diet), 25.6% sucrose, and 16.4% protein.

Control diet was purchased from Laboratory Animal Center of Southern Medical University, China.

### Example 2

### Methods of Group and Treatment

All the mice were divided into three groups (n=5/group).

At the beginning of the experiment, 6 weeks old C57BL/6J mice (no need for germ-free) were gavaged with freshly cultured R. inulinivorans DSM 16841. Oral glucose tolerance tests (OGTT) were performed on 10 h fasted mice for blood glucose levels tests.

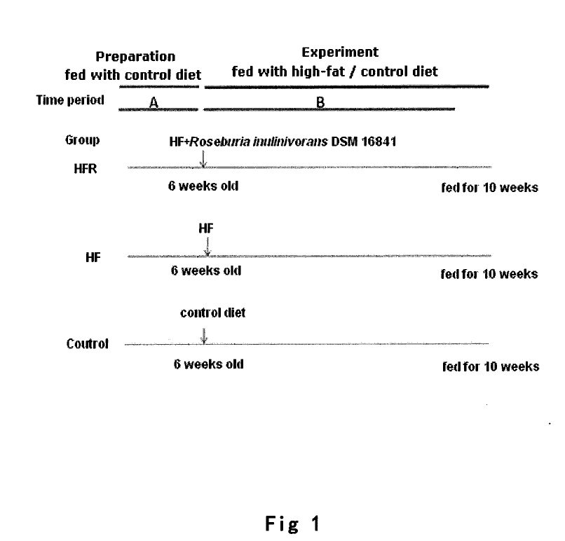

The detailed information and treatment methods were showed in FIG. 1.

After 3 weeks of acclimatization, these mice were housed in groups of five per cage in clean environment with free access to control food and water to reach 6 weeks old. Body weight of each mouse was monitored once a week while the food intake of each group was recorded every two days.

The 15 mice were divided into three groups (n=5/group). Their diets were modified as below. The control Group was fed with control diet, meanwhile the HF group and Roseburia-treated HFR group were fed with high-fat diet.

Each cage maintained clean during the experimental period. All the mice lived in their corresponding cages and gavaged in unified processes.

R. inulinivorans DSM 16841 was freshly suspended in culture medium weekly with the concentration 1×10⁷ cfu/mL, and the cultivation liquid compressed to 1×10⁸ cfu/mL. Then the strain was orally administered to the mice by gavage (0.15 mL/10 g body weight) every two days.

Body weight and daily food intake of each mouse were recorded during the experiment. After 5 and 10 weeks of treatments, oral glucose tolerance tests (OGTT) were performed and blood glucose levels of 10 h fasted mice were determined by using a glucose meter. Blood was collected from the tip of the tail vein both before and after glucose administration (60 min and 120 min).

### Example 3

### Results

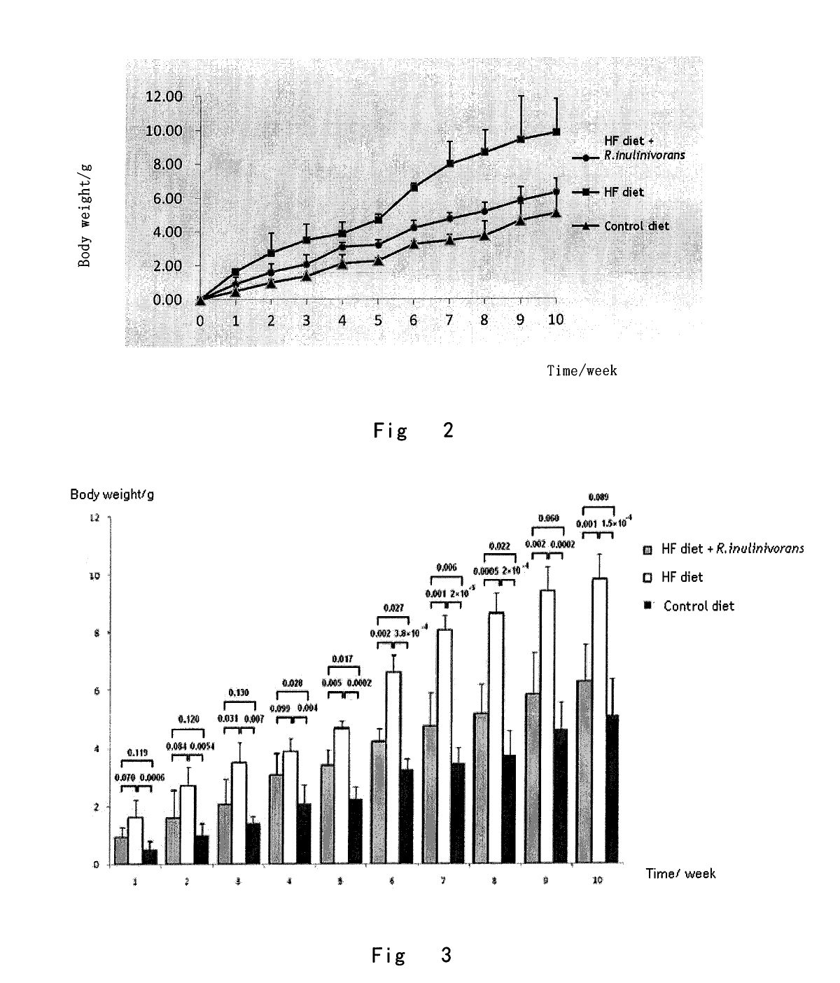

1. The average increases of body weight during the 10 weeks-treatment were shown in Table 1 and FIG. 2.

TABLE 1

| | Time (week) | | | | | | | | | | |
|---|---|---|---|---|---|---|---|---|---|---|---|
| Group | 0 | 1 | 2 | 3 | 4 | 5 | 6 | 7 | 8 | 9 | 10 |
| 1. HFR (g) | 0.00 | 0.92 | 1.60 | 2.06 | 3.10 | 3.20 | 4.22 | 4.74 | 5.18 | 5.86 | 6.30 |
| 2. HF (g) | 0.00 | 1.64 | 2.74 | 3.52 | 3.90 | 4.70 | 6.60 | 7.98 | 8.66 | 9.42 | 9.84 |
| 3. Control (g) | 0.00 | 0.50 | 1.00 | 1.40 | 2.10 | 2.27 | 3.27 | 3.50 | 3.77 | 4.65 | 5.10 |

2. The P values of average increase of body weight during the 10 weeks-treatment were shown in Table 2 and FIG. 3.

TABLE 2

| | Time (week) | | | | | | | | | |
|---|---|---|---|---|---|---|---|---|---|---|
| Group | 1 | 2 | 3 | 4 | 5 | 6 | 7 | 8 | 9 | 10 |
| P1-2 | 0.070 | 0.084 | 0.031 | 0.099 | 0.005 | 0.002 | 0.001 | 0.0005 | 0.002 | 0.001 |
| P1-3 | 0.119 | 0.120 | 0.130 | 0.028 | 0.017 | 0.027 | 0.006 | 0.022 | 0.060 | 0.089 |
| P2-2 | 0.0006 | 0.0054 | 0.007 | 0.004 | 0.0002 | 0.00038 | 0.00002 | 0.0002 | 0.0002 | 0.00015 |

It was shown that the body weight of HFR group was significantly lower than HF group from Table 1, Table 2, FIG. 2 and FIG. 3. The results suggested that R. inulinivorans DSM 16841 could help with the mitigation of obesity in mice.

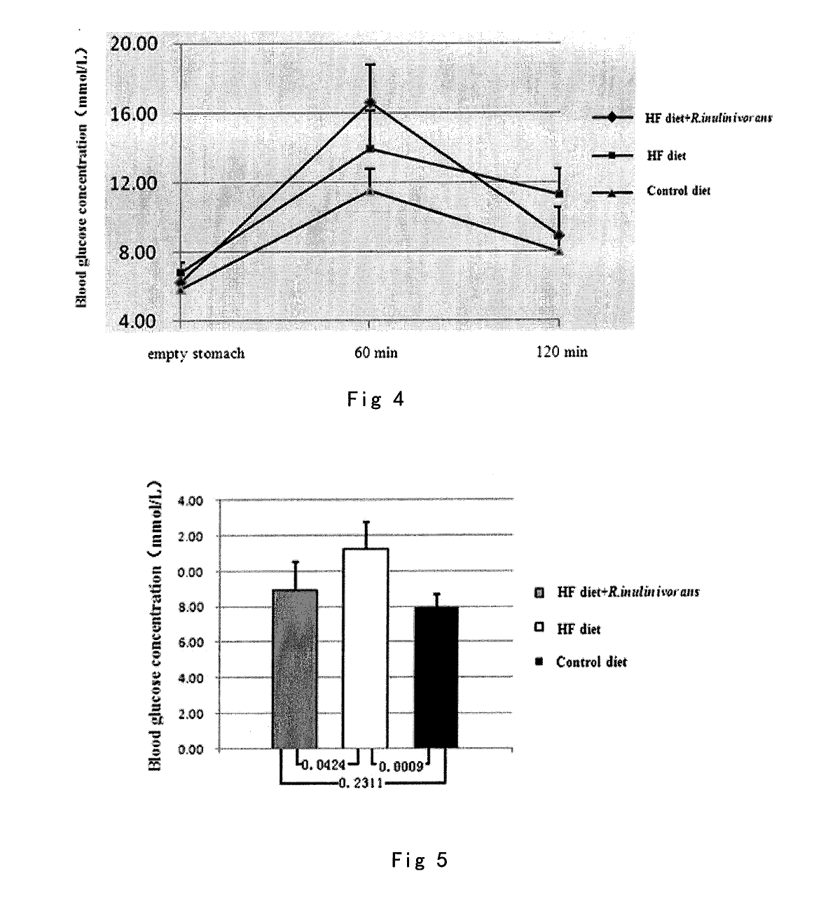

3. The blood glucose content of mice after 10 weeks was shown as Table 3, Table 4 and FIG. 4, FIG. 5.

TABLE 3

| | empty stomach | | |
|---|---|---|---|
| Group | (mmol/L) | 60 min (mmol/L) | 120 min (mmol/L) |
| 1. | 6.26 | 16.62 | 8.92 |
| 2. | 6.80 | 13.92 | 11.28 |
| 3. | 5.85 | 11.53 | 7.98 |

Table 3 and FIG. 4 indicated that the R. inulinivorans DSM 16841 could reduce the blood glucose of obese mice remarkably.

TABLE 4

| P Value | Empty stomach | 60 min | 120 min |
|---|---|---|---|
| P1-2 | 0.1238 | 0.0868 | 0.0424 |
| P1-3 | 0.3511 | 0.0009 | 0.2311 |
| P2-3 | 0.0641 | 0.0495 | 0.0009 |

Table 4 and FIG. 5 indicated that the R. inulinivorans DSM 16841 could improve the blood glucose tolerance of obese mice remarkably.

### Example 4

### Foodstuff Compositions Containing R. Inulinivorans DSM 16841

The components of the foodstuff compositions are shown in Table 5.

TABLE 5

| materials | percentage of content (%) |
|---|---|
| R. inulinivorans DSM 16841 | 0.5 |
| milk | 90.0 |
| sugar | 9.5 |

The components were mixed according to above mentioned ratio, stirred until mixed completely, and then preheated with high-pressure homogenization at 20 Mpa. The compositions were sterilized for 5-10 minutes at 90° C., and inoculated when the temperature cooled to 40-43° C. The amount of inoculation was 1-100×10⁶ cfu R. inulinivorans DSM 16841/g.

### Example 5

### Pharmaceutical Compositions Containing R. inulinivorans DSM 16841

The components of the foodstuff compositions are shown in Table 6.

TABLE 6

| materials | percentage of content (%) |
|---|---|
| R. inulinivorans DSM 16841 | 1.0% |
| lactose | 2.0% |
| yeast extract | 2.0% |
| peptone | 1.0% |
| pure water | 94.0% |

Lactose, yeast extract, peptone, and pure water were mixed according to above mentioned ratio, and preheated to 60-65° C. with high-pressure homogenization at 20 Mpa. The mixture was sterilized for 20-30 minutes at 90° C. R. inulinivorans DSM 16841 (1-50×10⁶ cfu/mL) was inoculated to the mixture when the temperature of the mixture was cooled to 36-38° C. After the resulting mixture fermented until pH turned to 6.0, it was centrifuged and freeze-dried to reach moisture content of less than 3% to prepare R. inulinivorans DSM 16841 bacteria freeze-dried material. 0.5 g R. inulinivorans DSM 16841 freeze-dried material was then mixed with equal amount of malto dextrin, and encapsulated to form a pharmaceutical composition containing R. inulinivorans DSM 16841.

All documents mentioned in this invention are cited as a reference, just as each one is a separate reference to literature as a reference. While the present invention has been particularly shown and described with reference to particular examples thereof, it will be understood by those skilled in the art that various changes in form and details may be conceived therefrom without departing from the spirit and scope of the present invention as defined by the appended claims.

1.-10. (canceled)

11. A method for treating or preventing obesity or reducing body weight, comprising administering to a subject in need thereof a bacterial strain selected from the Roseburia family.

12. The method of claim 11, wherein the bacterial strain is Roseburia inulinivorans.

13. The method of claim 11, wherein the bacterial strain is Roseburia inulinivorans DSM 16841.

14. A method for reducing blood glucose or improving tolerance of blood glucose, comprising administering to a subject in need thereof a bacterial strain selected from the Roseburia family.

15. The method of claim 14, wherein the bacterial strain is Roseburia inulinivorans.

16. The method of claim 14, wherein the bacterial strain is Roseburia inulinivorans DSM 16841.

17. A composition comprising a bacterial strain selected from the Roseburia family or metabolite thereof and a carrier.

18. The composition of claim 17, wherein the composition is a food composition.

19. The composition of claim 18, wherein the food composition is in a form selected from solid, diary, solution, powder, and suspension.

20. The composition of claim 18, wherein the bacterial strain is Roseburia inulinivorans.

21. The composition of claim 18, wherein the bacterial strain is Roseburia inulinivorans DSM 16841.

22. The composition of claim 17, wherein the composition is a pharmaceutical composition.

23. The composition of claim 22, wherein the composition is in the form selected from granule, capsule, tablet, powder, oral liquid, suspension, and emulsion.

24. The composition of claim 22, wherein the bacterial strain is Roseburia inulinivorans.

25. The method of claim 22, wherein the bacterial strain is Roseburia inulinivorans DSM 16841.
